(19) United States
(12) Patent Application Publication
(10) Pub. No.: US 2024/0105346 A1
(43) Pub. Date: Mar. 28, 2024

(54) OPTIMIZING MESSAGING TO SERVICE PRINCIPALS USING MACHINE LEARNING

(71) Applicant: Providence St. Joseph Health, Seattle, WA (US)

(72) Inventors: Wayne T. Foley, Seattle, WA (US); Adam Benjamin Smith-Kipnis, Seattle, WA (US); Lisa Dione Mason, Newcastle, WA (US); Billy Lee Jackson, Olympia, WA (US); Syneva Runyan, Kotzebue, AK (US); Ryan Alexander Untalan, San Francisco, CA (US); Yoomi Robin Kang, Prosper, TX (US)

(21) Appl. No.: 17/950,861

(22) Filed: Sep. 22, 2022

(51) Int. Cl. G16H 80/00; G06F 16/35; G06K 9/62

(52) U.S. Cl. CPC G16H 80/00; G06F 16/35; G06K 9/6256

(57) ABSTRACT

A message processing facility is described. The facility receives user input defining a textual message intended for an addressee service principal. Before sending the defined textual message, the facility analyzes the defined textual message to determine a textual message category to which the defined textual message belongs among a plurality of textual message categories.

### BACKGROUND

People increasingly use patient portals—such as via the web or smartphone apps—to engage electronically with their healthcare. It is common for patient portals to permit their users to send textual messages to their physicians and other medical providers.

### BRIEF DESCRIPTION OF THE DRAWINGS

FIG. 1 is a block diagram showing some of the components typically incorporated in at least some of the computer systems and other devices on which the facility operates.

FIG. 2 is a flow diagram showing a process performed by the facility in some embodiments to train a machine learning model to classify messages to service principals into message categories.

FIG. 3 is a data structure diagram showing sample contents of a category disposition table used by the facility in some embodiments to store a set of message categories and the disposition specified for each.

FIG. 4 is a flow diagram showing a process performed by the facility in some embodiments to process a draft message prepared by a user that is initially addressed to a service principal.

FIG. 5 is a display diagram showing sample contents of a display presented by the facility as a starting point for preparing a message to a service principal.

FIG. 6 is a display diagram showing sample contents of a display presented by the facility in some embodiments in response to the user activating email control 513 for a particular service principal.

FIG. 7 is a display diagram showing sample contents of a display presented by the facility in some embodiments in response to the user activating the next control 602.

FIG. 8 is a display diagram showing sample contents of a display presented by the facility in some embodiments after a user has entered the message.

FIG. 9 is a display diagram showing sample contents of a display presented by the facility in some embodiments in response to the user activating the next control 806.

FIG. 10 is a display diagram showing sample contents of a display presented by the facility in some embodiments in response to the user operating control 921.

FIG. 11 is a display diagram showing sample contents of a display presented by the facility in some embodiments to permit a user to select the appropriate category for a message if predicting the message category automatically is unsuccessful, such as where it produces no category predictions with an adequate confidence level.

FIG. 12 is a display diagram showing sample contents of a display presented by the facility in response to predicting that the category of a draft message is personal message.

FIG. 13 is a display diagram showing sample contents of a display presented by the facility in some embodiments after the user operates control 1232.

FIG. 14 is a display diagram showing sample contents of a display presented in some embodiments to select an addressee for a message in response to a user operating send message control 590 shown in FIG. 5.

### DETAILED DESCRIPTION

The inventors have identified significant disadvantages of the conventional ways in which patient portals handle textual messages from patients to physicians and other medical providers. First, as patients have discovered the ability to send textual messages to physicians, the volume of these messages has ballooned. This increase in volume has impacts on the addressee physicians such as: increased time processing messages and total workload; reduced ability to respond promptly and appropriately to messages that are urgent and important; and reduced average hourly revenue, and in some cases total revenue.

Second, many messages are directed to physicians that they are poorly-suited to answer, either because these messages pose questions that can only be effectively addressed by people in other categories—such as technical support, or in that they are ill-advised to use the patient portal to send to anyone—such as personal messages.

Third, the messages received by physicians are commonly distinguished only by the time they were sent, such the physician must open and read every message before being able to make decisions about answering it, ignoring it, forwarding or otherwise delegating it, taking some other action in response, etc.

In response to recognizing these disadvantages, the inventors have conceived and reduced to practice a software and/or hardware facility for optimizing messaging to service principals such as physicians using machine learning (“the facility”).

In some embodiments, the facility trains and applies one or more machine learning models to classify each textual message generated for a service principal by a user. In some embodiments, the facility classifies some messages into one of more categories that cause the facility to recommend that the user perform a self-service operation in place of sending the message. For example, in some embodiments, the facility determines that a particular message is in a category of seeking an appointment with a service principal to whom the message is addressed. In response to this categorization, the facility recommends to the user that the user perform a self-service process for scheduling the appointment directly in place of sending the message.

In some embodiments, the facility classifies some messages into one or more categories that cause the facility to recommend or unilaterally impose a new addressee for the message who is not a service principal. For example, in some embodiments, the facility determines that a particular message is in a category of seeking technical assistance with the patient portal. In response to this categorization, the facility redirects the message to a technical support worker, so that it is not sent to the service principal. In various embodiments, the facility similarly redirects messages in certain categories to a variety of other people, including service assistants such as medical assistants.

In some embodiments, the facility classifies some messages into particular categories in a group of categories that are appropriate for a service principal and that each have different character from the perspective of a service principal that cause the facility to distinguish among the messages assigned to these different categories for processing by the service principal to whom they are addressed. For example, in some embodiments, the facility determines that a particular message is in one of the following categories appropriate for a service principal: medical question; prescription question; test results question; or after-visit question. In response to classification into any of these categories, the facility delivers the message to the addressee service principal, with an indication of the assigned category. The service principal can then process receive messages on the basis of the category of each, such as by choosing a particular category of messages to review together; delegate to another person; defer, archive, or delete; etc.

In some embodiments, the facility classifies some messages into one or more categories that cause the facility to discourage the user from sending the message. For example, in some embodiments, the facility determines that a particular message is in a category of personal messages. In response to this categorization, the facility indicates that the user has chosen a poor modality for communicating a personal message, and invites the user to cancel the message. In some embodiments, the facility determines that a particular message is in a category of seeking medical advice about a medical issue; in response to this categorization, the facility recommends that the user schedule an appointment in place of sending the message to enable a thorough exchange about the medical issue upon which sound medical advice can be based. In some embodiments, the facility determines that a particular message is in a category of messages for which sending the message and/or receiving a response is a basis for charging the user; in response to this categorization, the facility warns the user that there may be a cost associated with proceeding with the message.

By operating in some or all of the ways described above, the facility reduces the volume of messages received by service principals, and provide information that places service principals in a better position to act more efficiently and effectively on the messages they do receive; and allows users to receive assistance in forms other than exchanging messages with the service principal, which may often prove more helpful to the users and may be more remunerative for the service principals.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by reducing the volume of messages sent to service principals, the facility reduces the level of memory resources needed to store these messages, as well as the level of processor resources needed to perform their substantive processing and the attendant user interface interactions, freeing up these resources for increased or improved use for other purposes, or permitting the devices involved to be replaced with lower-capacity, less-expensive versions.

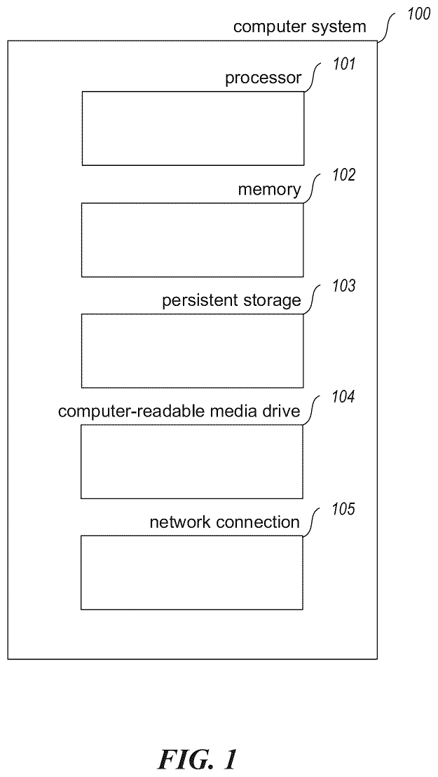

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102 for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

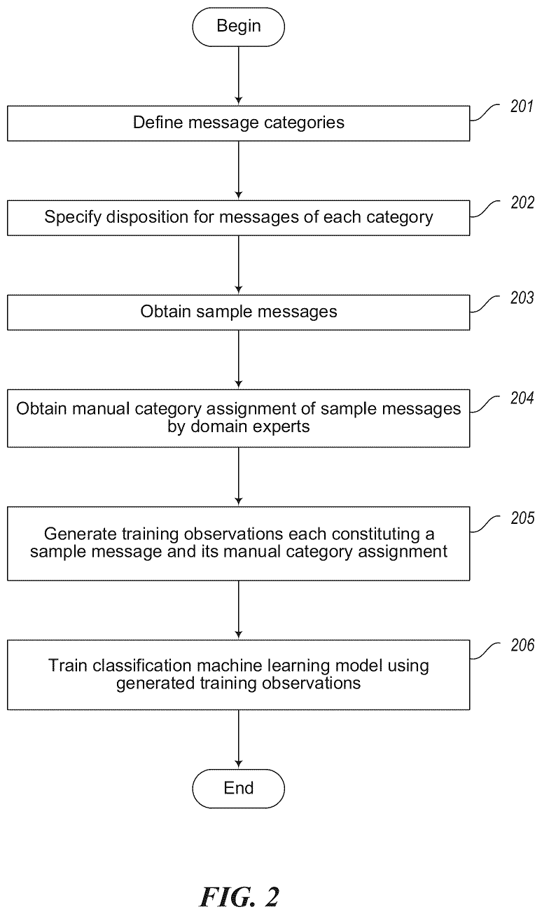

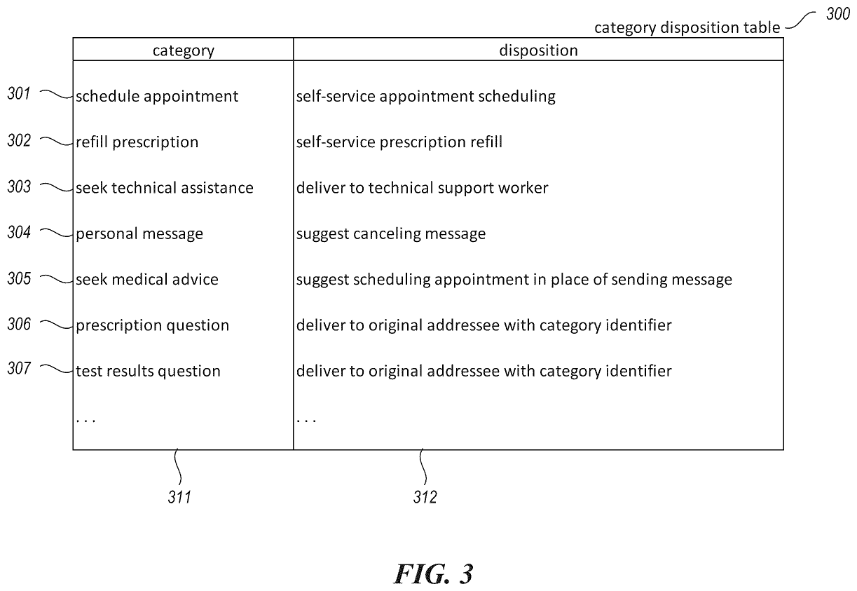

FIG. 2 is a flow diagram showing a process performed by the facility in some embodiments to train a machine learning model to classify messages to service principals into message categories. In act 201, the facility defines a set of message categories that tend to occur among messages to service principals, and have similar preferred dispositions. A set of message categories shown in FIG. 3 is discussed in greater detail below. In act 202, the facility specifies a disposition for messages of each of the categories defined in act 201. These, too, are shown in FIG. 3. In act 203, the facility obtains a set of sample messages. In some embodiments, the sample messages are actual messages written for and/or sent to service principals in a service area to which the facility will be applied, such as physicians, physicians in a certain practice area, etc. In some embodiments, the facility obtains sample messages that are prepared exclusively for training the model. In act 204, the facility obtains a manual category assignment of each of the sample messages obtained in act 203 by domain experts. In some embodiments, the domain experts are service principals of the type for which the facility will operate, or other people who work closely with such service principals. In act 205, the facility generates training observations each constituting one of the sample messages obtained in act 203 and the category manually assigned to it in act 204. In act 206, the facility uses the training observations generated in act 205 to train a classification machine learning model. In particular, in these training observations, the facility treats the sample message as an independent variable value, and its manual category assignment as a dependent variable value.

In various embodiments, the facility uses a variety of machine learning model types. In some embodiments, the facility uses a transformer-based machine learning model, such as those described in any of the following, each of which is hereby incorporated by reference in its entirety: “BERT” (available at huggingface.co/docs/transformers/model_doc/bert); J. Devlin, M. W. Chang, K. Lee, K. Toutanova, “BERT: Pre-training of Deep Bidirectional Transformers for Language Understanding,” arxiv: 1810.04805 (available at arxiv.org/abs/1810.04805); B. Portelli, “DiLBERT (Disease Language BERT)” (available at huggingface.co/beatrice-portelli/DiLBERT); W. Siblini, M. Challal, C. Pasqual, “Delaying Interaction Layers in Transformer-based Encoders for Efficient Open Domain Question Answering,” arxiv: 2010.08422 (available at arxiv.org/abs/2010.08422); and K. Roitero, B. Portelli, M. H. Popescu and V. D. Mea, “DiLBERT: Cheap Embeddings for Disease Related Medical NLP,” in IEEE Access, vol. 9, pp. 159714-159723, 2021, doi: 10.1109/ACCESS.2021.3131386. (available at ieeexplore.ieee.org/document/9628010). In cases where the present application conflicts with the document incorporated by reference, the present application controls. After act 206, this process concludes.

Those skilled in the art will appreciate that the acts shown in FIG. 2 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

FIG. 3 is a data structure diagram showing sample contents of a category disposition table used by the facility in some embodiments to store a set of message categories and the disposition specified for each. The table 300, populated by the facility using the process shown in FIG. 2 and discussed above, is made up of rows, such as rows 301-307, each corresponding to a different category. Each row is divided into column as follows: a category column 311 identifying the category to which the row corresponds, and a disposition column 312 specifying the disposition for messages of the category to which the row corresponds. For example, row 301 specifies that, for a “schedule appointment,” the specified disposition is to direct a user to perform a self-service process for scheduling an appointment.

While FIG. 3 and each of the table diagrams discussed below show a table whose contents and organization are designed to make them more comprehensible by a human reader, those skilled in the art will appreciate that actual data structures used by the facility to store this information may differ from the table shown, in that they, for example, may be organized in a different manner; may contain more or less information than shown; may be compressed, encrypted, and/or indexed; may contain a much larger number of rows than shown, etc.

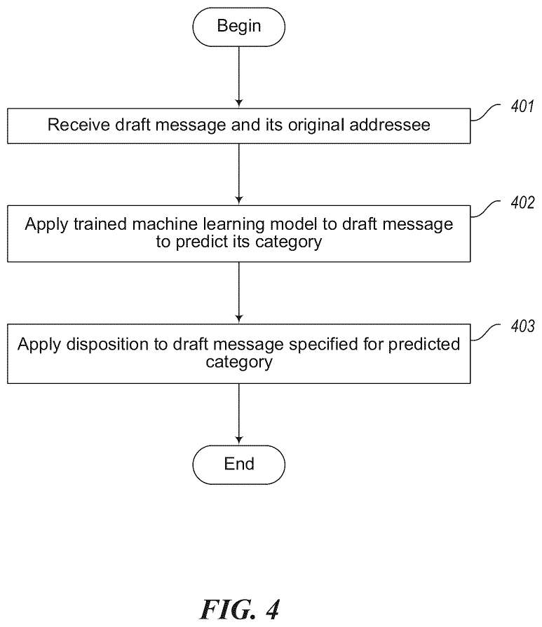

FIG. 4 is a flow diagram showing a process performed by the facility in some embodiments to process a draft message prepared by a user that is initially addressed to a service principal. In act 401, the facility receives the draft message and its original addressee. For example, in some embodiments, the user types this information into a web page or application form provided by the facility, and/or an electronic medical record (“EMR”) program. In act 402, the facility applies the trained machine learning model to the draft message received in act 401 to predict the category of the draft message. In some embodiments, the model returns a confidence level for each of the message categories indicating the likelihood that the message is a member of that category. In some embodiments, the facility selects as the category of a message any categories having a confidence level higher than a particular threshold, such as 90%. In some embodiments, the facility selects as the category of the message the category having the highest confidence level. In act 403, the facility applies to the draft message the disposition specified for the category predicted in act 402, such as the disposition identified by the category disposition table shown in FIG. 3. After act 403, this process concludes.

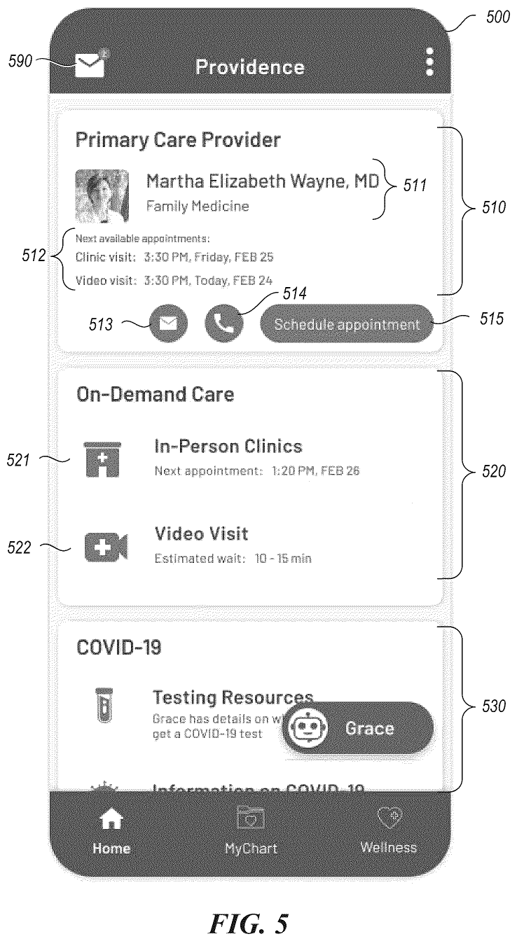

FIGS. 5-14 show sample contents of displays typically presented by the facility.

FIG. 5 is a display diagram showing sample contents of a display presented by the facility as a starting point for preparing a message to a service principal. The display 500 includes information 510 about a particular service principal, including identifying information 511, schedule information 512, messaging control 513, calling control 514, and appointment scheduling control 515. A user may operate these controls to prepare a message to the service principal; telephone the service principal; their office, or their assistant, or schedule an appointment with the service principal, respectively. The display also includes section 520 about available on-demand care options, including appointments 521 at in-person clinics, and appointments 522 for video visits. In some embodiments, this on-demand care will not necessarily be with the service principal identified in section 510. The display also includes a section 530 about a prevalent viral infection, including information about the infection, testing resources, etc. The display further contains an additional messaging control 590 that the user may operate to send a message to any of the service principals with whom the user has been enabled to communicate, such as physicians and other medical professionals for treating the patient.

While FIG. 5 and each of the display diagrams discussed below show a display whose formatting, organization, informational density, etc., is best suited to certain types of display devices, those skilled in the art will appreciate that actual displays presented by the facility may differ from those shown, in that they may be optimized for particular other display devices, or have shown visual elements omitted, visual elements not shown included, visual elements reorganized, reformatted, revisualized, or shown at different levels of magnification, etc.

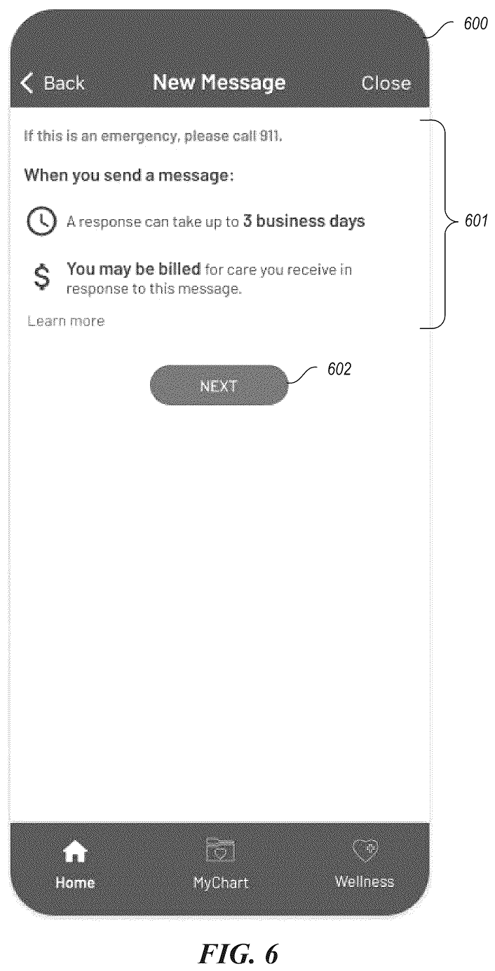

FIG. 6 is a display diagram showing sample contents of a display presented by the facility in some embodiments in response to the user activating messaging control 513 for a particular service principal. The display 600 presents information 601 establishing expectations for the message that is to be sent, including the amount of time it can take to receive a response, and the fact that sending the message may result in being charged. The display includes a next control 602 that the user can activate to proceed to prepare the message.

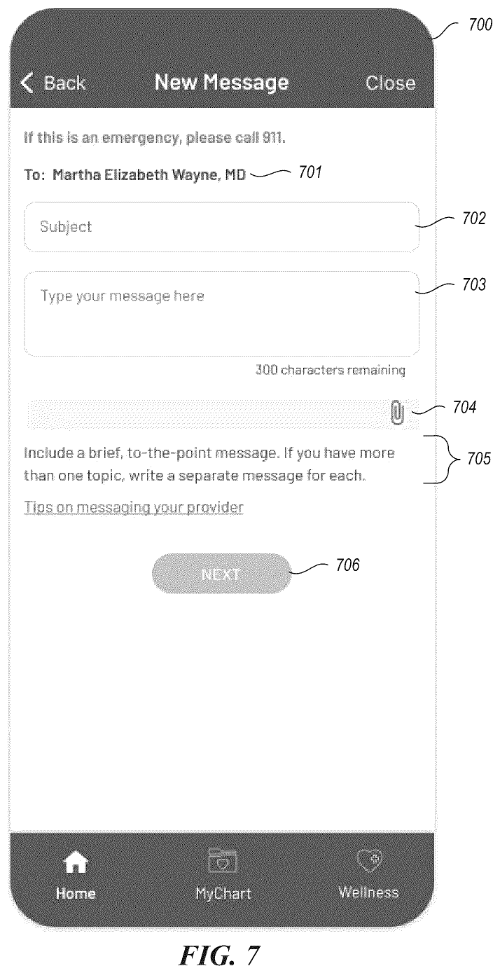

FIG. 7 is a display diagram showing sample contents of a display presented by the facility in some embodiments in response to the user activating the next control 602. The display 700 contains an indication 701 of the service principal to whom the message is addressed. It further contains field 702 for entering the subject of the message, field 703 for entering the body of the message, and attachment control 704 for attaching one or more attachments. The display further contains directions 705 about preparing the message, and a next control 706 that the user can activate after preparing the message.

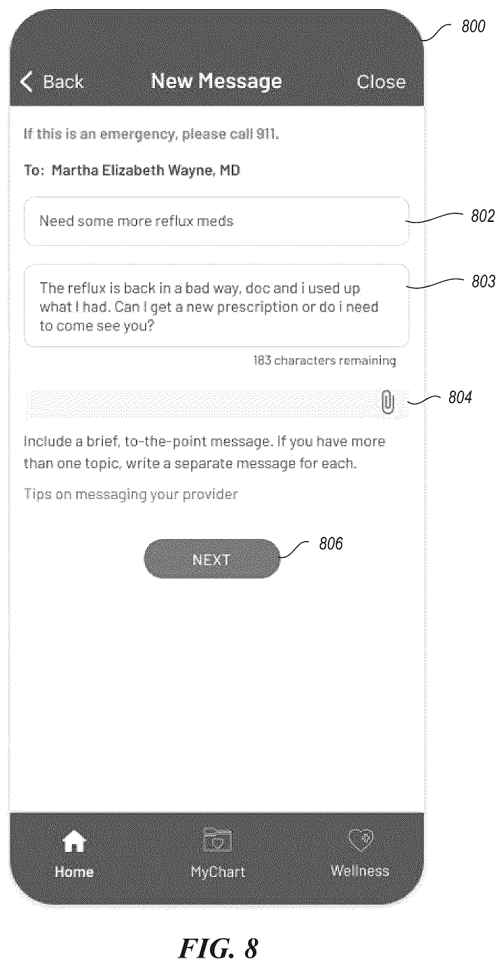

FIG. 8 is a display diagram showing sample contents of a display presented by the facility in some embodiments after a user has entered the message. In particular, the display 800 contains the subject in subject field 802, and a message body in message body 803. The next control 806 is now ungrayed, and operable by the user.

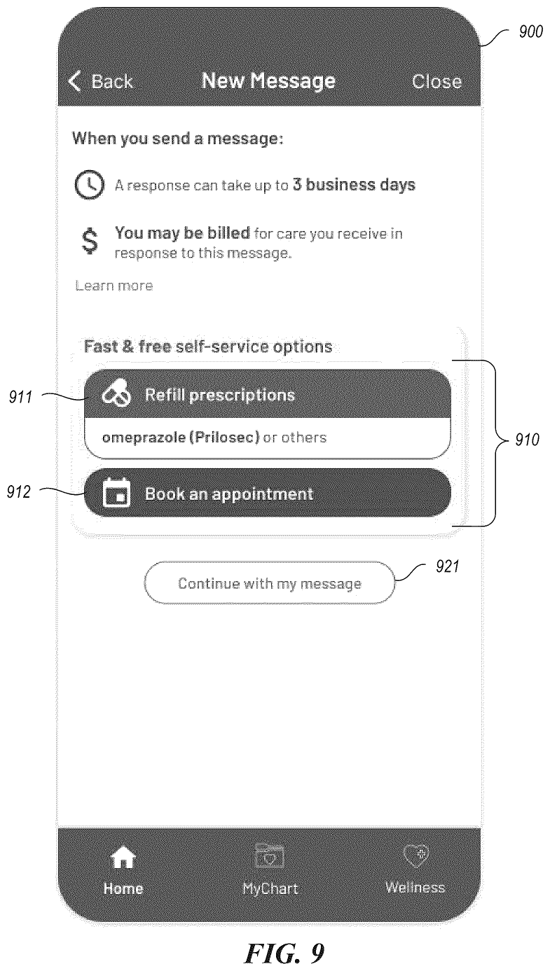

FIG. 9 is a display diagram showing sample contents of a display presented by the facility in some embodiments in response to the user activating the next control 806. In particular, display 900 contains information about self-service processes that the user can perform. In particular, the display includes a refill prescriptions control 911 that the user can operate in order to perform a self-services prescription refill operation, and an appointment booking control 912 that the user can operate in order to perform a self-service appointment booking process. In some embodiments, the facility has subjected the message shown in FIG. 8 to classifications, resulting in two categories: refill prescription and schedule appointment. Based upon the dispositions specified for these categories in rows 302 and 301 of the category disposition table, the facility has displayed these controls. The display 900 further includes a continue with message control 921 that the user can operate in order to continue with the process of preparing and sending the message.

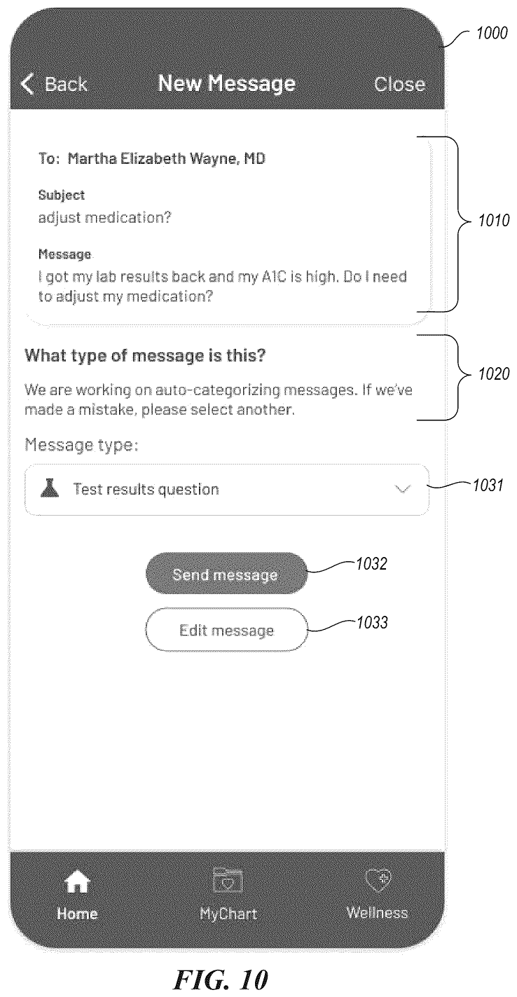

FIG. 10 is a display diagram showing sample contents of a display presented by the facility in some embodiments in response to the user operating control 921. The display 1000 shows message contents 1010, as well as an inquiry 1020 about requesting validation of the category 1031 predicted for the message by the facility. It should be noted that message 1010 has been changed relative to message 803 shown in FIG. 8, and thus has produced a different category prediction 1031—test results question—than that earlier message. If the user wishes to send the message with category 1031, the user can activate send message control 1032. If the user wishes to further edit the message first, the user can activate control 1033. If the predicted category 1031 is incorrect, the user can click on the predicted category to display a list of available categories and select the appropriate one.

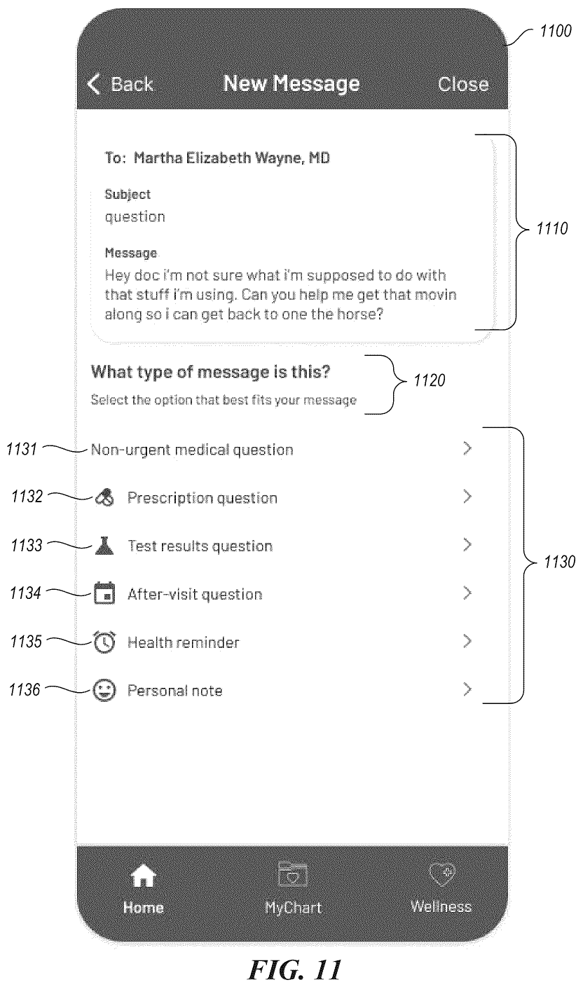

FIG. 11 is a display diagram showing sample contents of a display presented by the facility in some embodiments to permit a user to select the appropriate category for a message if predicting the message category automatically is unsuccessful, such as where it produces no category predictions with an adequate confidence level. The display 1100 includes a third message 1110; a prompt 1120 to select message category; and available categories 1130 from among which the user can choose.

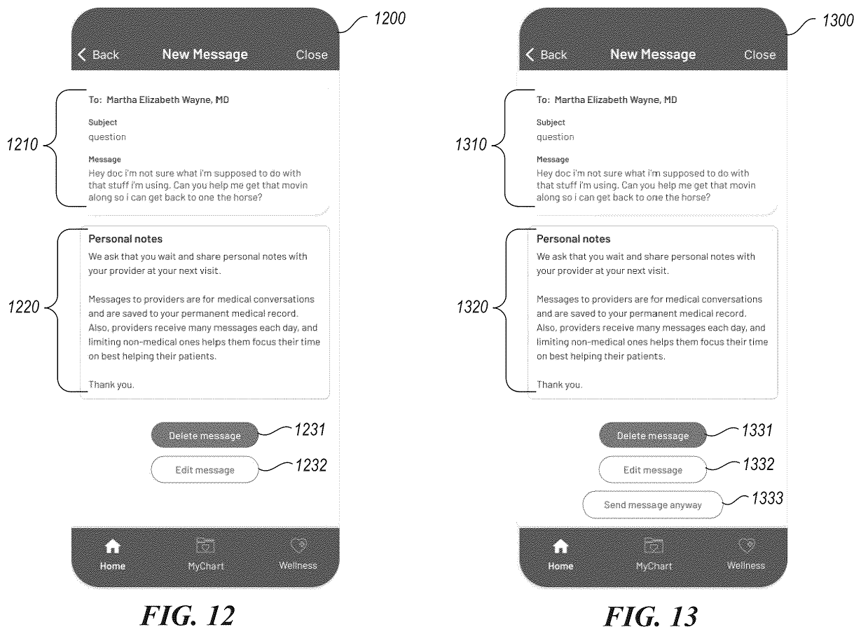

FIGS. 12 and 13 show a process performed by the facility to discourage the user from sending a message predicted to be in a disfavored category, here personal messages.

FIG. 12 is a display diagram showing sample contents of a display presented by the facility in response to predicting that the category of a draft message is personal message. Based upon the disposition specified for this message category by row 304 of the category disposition table—suggest canceling message—the facility presents information 1220 recommending against sending a personal note as a service principal message. The display also includes a control 1231 which the user can operate to delete this message, and a control 1232 that the user can operate to edit this message. If the user clicks on the edit message control 1232, the facility alters the display as shown in FIG. 13.

FIG. 13 is a display diagram showing sample contents of a display presented by the facility in some embodiments after the user operates control 1232. It can be seen that by comparing display 1300 shown in FIG. 13 to display 1200 shown in FIG. 12, that the facility has added a send message anyway control 1333. The user can operate this send message anyway control 1333 to send the message despite it being in the personal message category.

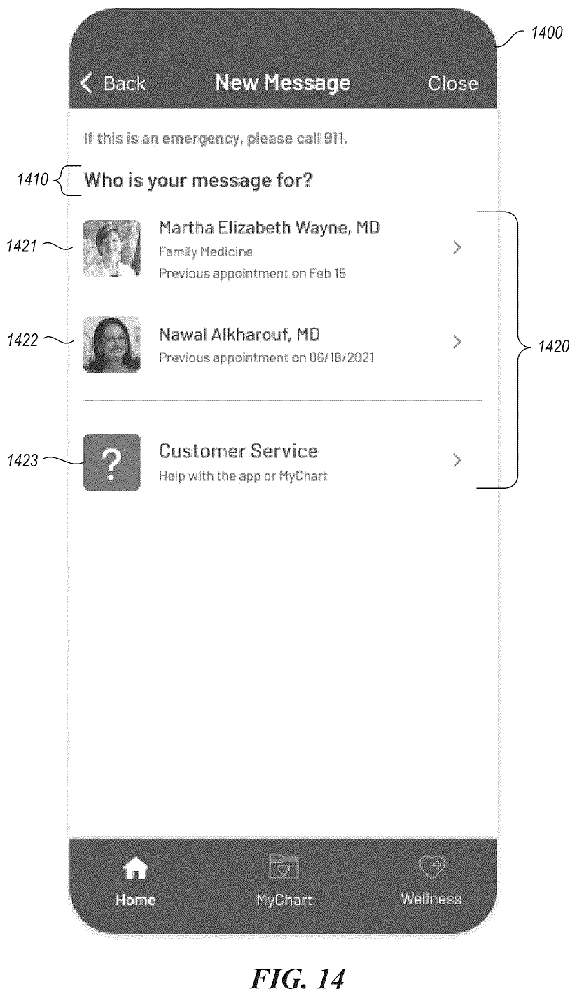

FIG. 14 is a display diagram showing sample contents of a display presented in some embodiments to select an addressee for a message in response to a user operating send message control 590 shown in FIG. 5. The display 1400 includes potential addressees 1420, including two service principals 1421 and 1422—such as the user's medical providers—as well as a customer service message queue 1423 for help with the patient portal. The user can select any of these addressees to begin the process of preparing and sending a message shown in FIGS. 6-10.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, comprising:
receiving user input defining a textual message intended for an addressee service principal;
before sending the defined textual message, subjecting the textual message to a machine learning classification model trained to predict textual message category based on textual message contents to obtain for the defined textual message a textual message category among a plurality of textual message categories;
from among a plurality of mappings each from one of the plurality of textual message categories to a textual message disposition strategy, accessing a mapping from the determined textual message category; and
performing the textual message disposition strategy to which the accessed mapping maps the determined textual message category with respect to the defined textual message.

2. The method of claim 1 wherein the addressee service principal operates in a distinguished service domain,
further comprising:
accessing a plurality of sample textual messages each intended for an addressee service principal in the distinguished service domain;
for each of the plurality of sample textual messages:
accessing a category among the plurality of textual message categories manually assigned to the sample textual message;
constructing a training observation in which the sample textual message is an independent variable and the category manually assigned to the sample textual message is a dependent variable; and
training the machine learning classification model using the constructed training observations.

3. The method of claim 1 wherein performing the textual message disposition strategy comprises causing the defined textual message to be delivered to the addressee service principal with information identifying the determined textual message category.

4. The method of claim 1 wherein performing the textual message disposition strategy comprises causing the defined textual message to be delivered to a person who is not a service principal.

5. The method of claim 1 wherein performing the textual message disposition strategy comprises:
initiating a self-service process corresponding to the determined textural message category; and
discarding the defined message without delivering it to any person.

6. The method of claim 1 wherein performing the textual message disposition strategy comprises triggering a reimbursable service assignment performed by the addressee service principal.

7. The method of claim 1 wherein performing the textual message disposition strategy comprises:
causing to be displayed:
an advisory that the textual messaging mode is ill-suited to textual messages of the determined textual message category, and
a control for canceling the message;
receiving user input activating the control; and
in response to receiving the user input activating the control, discarding the defined message without delivering it to any person.

8. One or more memory devices collectively storing a trained machine learning model data structure, the data structure comprising:
values of learnable parameters defining a machine learning model trained to predict a textual document category among a plurality of textual document categories to which a textual document belongs based upon contents of the textual document,
such that the contents of the data structure are usable to predict a distinguished textual document category to which a distinguished textual document belongs based upon contents of the distinguished textual document.

9. The one or more memory devices of claim 8, the data structure further comprising:
for each of the plurality of textual document categories, information specifying a textual message disposition strategy for textual messages predicted to belong to the textual document category,
such that the contents of the data structure are further usable to select a textual message disposition strategy to perform for the distinguished textual document based on the distinguished textual document category.

10. One or more instances of computer-readable media collectively having contents configured to cause a computing system to perform a method, the method comprising:
receiving user input defining a textual message intended for an addressee service principal; and
before any sending of the defined textual message, analyzing the defined textual message to determine a textual message category to which the defined textual message belongs among a plurality of textual message categories.

11. The one or more instances of computer-readable media of claim 10 wherein the analyzing comprises subjecting the defined textual message to a machine learning classification model trained to predict textual message category based on textual message contents.

12. The one or more instances of computer-readable media of claim 11 wherein the addressee service principal operates in a distinguished service domain, the method further comprising:
accessing a plurality of sample textual messages each intended for an addressee service principal in the distinguished service domain;
for each of the plurality of sample textual messages:
accessing a category among the plurality of textual message categories manually assigned to the sample textual message;
constructing a training observation in which the sample textual message is an independent variable and the category manually assigned to the sample textual message is a dependent variable; and
training the machine learning classification model using the constructed training observations.

13. The one or more instances of computer-readable media of claim 10, the method further comprising:
from among a plurality of mappings each from one of the plurality of textual message categories to a textual message disposition strategy, accessing a mapping from the determined textual message category; and
performing the textual message disposition strategy to which the accessed mapping maps the determined textual message category with respect to the defined textual message.

14. The one or more instances of computer-readable media of claim 13 wherein performing the textual message disposition strategy comprises causing the defined textual message to be delivered to the addressee service principal with information identifying the determined textual message category.

15. The one or more instances of computer-readable media of claim 13 wherein performing the textual message disposition strategy comprises causing the defined textual message to be delivered to a person who is not a service principal.

16. The one or more instances of computer-readable media of claim 13 wherein performing the textual message disposition strategy comprises:
initiating a self-service process corresponding to the determined textural message category; and
discarding the defined message without delivering it to any person.

17. The one or more instances of computer-readable media of claim 13 wherein performing the textual message disposition strategy comprises triggering a reimbursable service assignment performed by the addressee service principal.

18. The one or more instances of computer-readable media of claim 13 wherein performing the textual message disposition strategy comprises:
causing to be displayed:
an advisory that the textual messaging mode is poorly-suited to textual messages of the determined textual message category, and
a control for canceling the message;
receiving user input activating the control; and
in response to receiving the user input activating the control, discarding the defined message without delivering it to any person.
